(19) United States
(12) Patent Application Publication
(10) Pub. No.: US 2025/0093321 A1
(43) Pub. Date: Mar. 20, 2025

(54) FIELD DEPLOYABLE MINIATURE SENSOR FOR CONTINUOUS AND IN-SITU MONITORING OF SOIL NUTRIENT

(71) Applicant: University of New Hampshire, Durham, NH (US)

(72) Inventors: MD Shaad Mahmud, Durham, NH (US); Wilfred Wollheim, Durham, NH (US)

(21) Appl. No.: 18/883,566

(22) Filed: Sep. 12, 2024

Related U.S. Application Data
(60) Provisional application No. 63/582,720, filed on Sep. 14, 2023.

(51) Int. Cl. G01N 33/24; G01N 1/44; G01N 21/31

(52) U.S. Cl. CPC G01N 33/24; G01N 1/44; G01N 21/31

(57) ABSTRACT

A system for in-situ measurement of substances in soil includes a probe having an inner portion movably coupled to, and positioned inside of, an outer portion. The outer portion includes a lower end adapted for insertion into soil, and for isolating a soil sample in a heating zone. One or more gas fittings are arranged to provide gas to the heating zone, to remove gas from the heating zone, or both. The inner portion being a smaller dimension in width than the outer portion, and the inner portion and the outer portion are arranged to provide one or more channels for the passage of air, gaseous products, or both. A heating device is configured to apply heat to the soil sample in the heating zone to release gaseous products. A sensor is arranged to detect substances in the gaseous products released, the substances being indicative of nutrients in the soil.

### RELATED APPLICATION

This application claims the benefit of U.S. Provisional Application No. 63/582,720, filed on Sep. 14, 2023.

The entire teachings of the above application are incorporated herein by reference.

### BACKGROUND

There is a global effort to find practices for food production that can sustainably feed the human population, which is expected to surpass ten billion people by 2050. The Haber-Bosch process enabled the use of inexpensive fertilizers to increase food production, with a >600% increase in their use in the past 50 years. Some key indicators of soil fertility and soil health are soil organic matter (SOM) content. As global demand for food continuously increases, and as land degradation frequently occurs, there is an urgent need to understand the dynamic changes of SOM in soils for better agricultural practices and environmental sustainability. However, such need is heavily hampered by the inability to make site-specific, low-cost, high-frequency measurements of those key parameters (e.g., soil organic carbon (SOC), total nitrogen (TN), carbon to nitrogen ratio (C/N), etc.) that could vary significantly both at the temporal and spatial scale.

### SUMMARY

Embodiments relate to a system for in-situ measurement of substances in soil includes a probe having an outer portion and an inner portion. The inner portion is movably coupled to the outer portion and is positioned inside the outer portion. The outer portion has a lower end for insertion into the soil, and for isolating a soil sample in a heating zone. The system includes one or more gaseous fittings arranged to provide gas to the heating zone, or to remove gas from the heating zone (for example, by extracting gas from the heating zone), or both. The inner portion and the outer portion are arranged to provide one or more channels for the passage of air, gaseous products, or both. The system includes a heating device which is configured to apply heat to the soil sample in the heating zone to release gaseous products. The system also includes a sensor arranged to detect substances in the gaseous products released, and the substances are indicative

The outer portion of the probe can be a cylindrical tube, and the inner portion of the probe a cylindrical rod. In some embodiments, both the outer portion and the inner portion are cylindrical tubes.

The system can include a portable power supply configured to supply power to the heating device.

The system can be integrated in a portable hand-held device.

The system can be mounted on an unmanned aerial vehicle, and further including a hydraulic press to insert the system into the soil.

The system can include a gaseous inlet and a gaseous outlet are positioned such that a channel between the gaseous inlet and the heating zone is created, and a separate channel between the heating zone and the gaseous outlet is created.

In the system, the inner portion can be of a smaller dimension than the outer portion, such that a channel is created between the inner portion and the outer portion for the passage of air and gaseous products.

The system can include a mesh screen attached radially around the inner portion above the heating device such that the mesh screen creates a barrier preventing the soil from entering the channels between the inlet and the heating zone, and the heating zone and the outlet.

The system can include a catalyst converter operably coupled to the gaseous outlet. For example, the catalyst converter converts Carbon-containing gas species and Nitrogen-containing gas species in the gas products into carbon dioxide (CO₂) and molecular nitrogen (N₂) respectively. They system can also include a sensor node operably coupled to the catalyst converter, the sensor node including the sensor arranged to detect the chemical compounds and an environmental sensor arranged to detect CO₂ content in air. For example, the sensor node can be configured to detect an amount of CO₂ and an amount of N₂.

The inner and outer portions can be movable between a first position and a second position. In the first position, the system is insertable into the soil utilizing the lower end of the outer portion such that the outer portion and the inner portion are both at the same depth in the soil. In the second position, the outer portion is extendable deeper into the soil, leaving the inner portion at a shallower depth in the soil and capturing the soil sample around the heating device. The system can include an upper end of the inner portion extending through an opening at a top end of the outer portion as the outer portion extends deeper into the soil. The system may also include a locking mechanism coupled to the inner portion and the outer portion that is configured to lock the system in the first position or the second position.

In the system, the inner portion can extend through the opening at the top surface of the outer portion has markings on it which indicate a difference in depth between the outer portion and inner portion, such that a volume of the soil filled area can be calculated.

Another embodiment is directed toward a method for in-situ measurement of substances in soil. The method includes inserting a probe into soil, isolating a soil sample in a heating zone, applying heat to the soil sample in the heating zone to release gas products, and detecting substances in the gas products released, the substances being indicative of nutrients in the soil.

The probe can include an inner portion movably coupled to any position inside an outer portion. The outer portion can include a lower end adapted for insertion into the soil, and for capturing the soil sample in the heating zone, as well as one or more gaseous fittings arranged to provide gas to the heating zone or to remove gas from the heating zone, or both. In the method, the inner portion can be of a smaller dimension in width than the outer portion, and the inner portion and the outer portion are arranged to provide one or more channels for the passage of air, gaseous products, or both.

In the method, inserting the probe into the soil includes inserting the inner and outer portions of the probe into the soil to a first depth, and further inserting the outer portion into the soil to a second depth.

In the method, at least one of the inner portion and the outer portion is inserted into the soul utilizing a hydraulic press.

In the method, detecting the chemical compounds includes catalytically converting Carbon-containing gas species and Nitrogen-containing gas species in the gas products into CO₂ and N₂, respectively, and detecting the CO₂ and N₂.

In the method, the inner and outer portions may be movable between a first position and a second position. In the first position, the method includes inserting the system into the soil utilizing the lower end of the outer portion such that the outer portion and the inner portion are both at the same depth in the soil, and in the second position, extending the outer portion deeper into the soil, leaving the inner portion at a shallower depth in the soil and capturing the soil sample around the heating device, and extending an upper end of the inner portion through an opening at a top end of the outer portion as the outer portion extends deeper into the soil. The method may also include locking the system in the first position or the second position using a locking mechanism coupled to the inner portion and the outer portion.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1D show an embodiment of a system for in-situ measurement of carbon and nitrogen inserted into soil.

FIG. 2 shows a graph representing testing results of CO₂ concentration in soil after heat is applied.

FIG. 3A shows an embodiment of a system for in-situ measurement of carbon and nitrogen inserted into soil.

FIG. 3B shows an example heating element for use with embodiments of the invention.

FIGS. 4A-4D show an embodiment for a system for in-situ measurement of carbon and nitrogen in soil.

FIGS. 5A-5B are side and sectional views, respectively, of a heating probe according to an embodiment of the invention.

FIG. 6 shows an exploded perspective view of the heating probe of FIG. 5A.

FIGS. 7A-7B are side and sectional views, respectively, of a heating probe in a standard configuration according to an embodiment of the invention.

FIGS. 7C-7D are side and sectional views, respectively, of the heating probe of FIG. 7A in a measuring configuration according to an embodiment of the invention.

FIGS. 8A-8D show a heating probe with handles, and a rotational locking mechanism, according to an embodiment of the invention.

### DETAILED DESCRIPTION

A description of example embodiments follows.

Portable soil sensors have been developed to provide quick and accurate measurements of soil properties, such as nutrients, pH, and salinity, in the field or on-site. However, there are still some challenges and limitations that can be addressed through new and novel innovations.

For example, one of the challenges is limited accuracy and precision. Current portable soil sensors may not provide highly accurate and precise measurements, especially in heterogeneous and complex soil environments. New innovations can be developed to improve the sensor performance, such as using new sensing technologies, integrating machine learning algorithms, and developing calibration and validation methods.

There is also lack of versatility and compatibility. Current portable soil sensors may not be versatile enough to measure a wide range of soil properties and to be compatible with different soil types and conditions. New innovations can be developed to increase the versatility and compatibility of the sensors, such as developing multi-functional sensors, integrating wireless and Internet of Things (IoT) technologies, and using modular and customizable designs.

In addition, current portable soil sensors may not be user-friendly and accessible to farmers, landowners, and other stakeholders who need to use them. New innovations can be developed to improve the usability and accessibility of the sensors, such as developing intuitive and interactive interfaces, providing real-time and actionable feedback, and creating user communities and knowledge networks.

Also, current portable soil sensors may not be sustainable and affordable in the long run, especially in developing countries and resource-limited settings. New innovations can be developed to improve the sustainability and affordability of the sensors, such as using renewable energy sources, designing low-cost and low-power sensors, and developing collaborative and open-source platforms.

Embodiments provided herein disclose a new and novel innovative system with high spatiotemporal can be developed to improve the accuracy, versatility, usability, and sustainability of portable soil sensors, and to enable more effective and efficient soil management practices that can benefit the environment, the economy, and the society.

It should be understood by a person of ordinary skill that although these disclosures refer to detecting the amount of soil and organic carbon in the soil, the embodiments described herein are well suited to detect a variety of alternative substances in the soil.

The amount of soil organic carbon (SOC) significantly impacts the quality of the soil and the rate of plant growth. It also plays a regulatory role in a variety of physical, chemical, and biological processes that occur in the soil environment. As a crucial indication of soil quality, it is linked to vital ecosystem services like nutrient cycling and storage, pollutant absorption and retention, and carbon dioxide sequestration. Carbon can be found in two different forms: inorganic and organic (IC and OC, respectively). In soil, IC is represented by carbon dioxide, carbonic acid, and its dissociation products. There is also calcite in the form of particles. The total amount of organic carbon in the world's soils is roughly 2344 gigaton, making them the giant terrestrial organic carbon pool. Even minute shifts in the amount of organic carbon in the soil can significantly affect the amount of carbon in the atmosphere. The fluxes of SOC change in response to a wide variety of potential natural and anthropogenic driving factors. As a result, it is necessary to develop a system that can estimate the amount of SOCs quickly and affordably to improve environmental monitoring, modeling, and precision agriculture.

Most of the time, the SOC content is measured in the laboratories. Combustion methods, chemical analysis, and spectroscopic methods are all common methodologies that can be used to estimate the amount of carbon in soil. However, standard laboratory studies to determine the content of SOC require a significant amount of time, are very expensive, and are unable to effectively depict the geographical and temporal fluctuations of SOC contents across large areas. Although, in-situ measurement of total soil carbon has been tested, this method suffers from tedious experimental setup and accuracy is susceptible to soil moisture and availability of the local spectrum data. Due to this, embodiments which provide an in-situ system capable of making a prediction in real time regarding the amount of OC that is contained within the soil are disclosed.

Embodiments provided herein make use of the soil combustion method. SOC decomposes at 400 degrees Celsius, while the decomposition of inorganic carbon takes place between 500 and 600 degrees Celsius. The loss-on-ignition (LOI) method is one common approach to measure SOC by combustion. In this technique, soil samples are burned at high temperatures, and the SOC content is calculated based on the weight loss. Soil is first air-dried in the laboratory to remove moisture and then heated at high temperatures in a metal container. The loss in mass after the heat is the indicator of soil organic matter. Then an elemental analyzer is used to measure OC. Elemental analyzers are expensive large pieces of equipment, and unsuitable for in-situ analysis. In addition, precise measurement is necessary for determining weight reduction. Therefore, embodiments provide for a direct measurement for CO₂ produced using a photoacoustic sensor to estimate SOC.

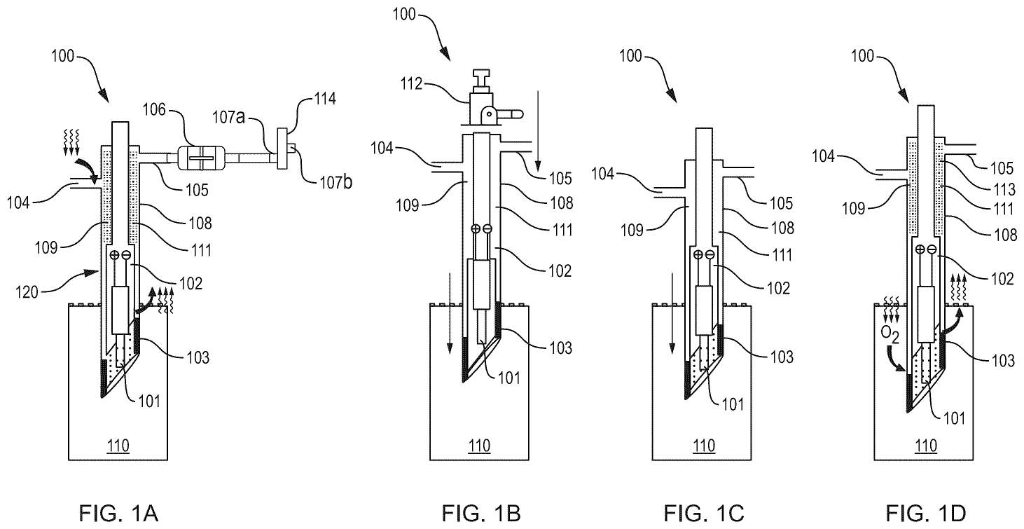

FIGS. 1A-1D illustrate an embodiment which is a cost-effective, high-precision sensing system 100 for detecting deep soil C/N with high spatiotemporal resolution. FIG. 1A shows the typical embodiment where the system 100 is inserted into the soil 110. The system 100 is configured to employ a simple heating-based decomposition method combined with a catalytic converter and gas detectors in a double-walled tube platform. The system includes a probe 120, which includes an inner tube 102 (e.g., an inner portion) movably coupled to, and positioned inside of, an outer tube 108 (e.g., and an outer portion). Inner and outer steel tubes 102 and 108 (in some embodiments, the inner portion may be a steel rod), respectively, provide the primary structural element for the system 100. Inside the outer tube 108, the inner tube 102 can move vertically. The inner tube 102 aids in the configuration of the system 100's ability to dig down to a specific depth of soil 110 and collect a predetermined volume of soil for burrowing. By mounting a heating rod 101 on the inner wall of the sliding inner tube 102, soil passing through the heating zone 103 can undergo continuous decomposition of all soil organic carbon (SOC) and total nitrogen (N). A portable power supply is used to power the heating rod 101. Changing power supply voltage and current regulates temperature of the rod 101. A gap 109 between the inner tube 102 and outer tube 108 provides oxygen for soil burning. When the heating rod 101 burns soil inside the heating zone 103, produced CO₂ moves up the tube through the gap.

A sensor node 114 is attached to the outer tube 108. The sensor node 114 has two CO₂ sensors in the inner 107a and outer 107b directions of the tube. The outer sensor 107b measures the air CO₂ content, and the inner sensor 107a is configured to provide a similar measurement prior to combustion. The concentration change inside the tube is determined based on the differential measurement of the inner and outer sensor after combustion. Additionally, the two-sensor configuration aids in identifying any sensor output anomalies.

The gaseous product composition (carbon monoxide (CO), CO₂, hydrocarbons, nitrous oxide (N₂O), nitrogen oxide (NOx), and NO₂) will depend on the oxygen availability during the heating process. To ensure sufficient oxygen supply, an air inlet 104 is installed on the outer tube 108 which creates a channel 109 to provide air to the heating zone 103, while an air outlet 105 is installed on the outer tube 108 to create a channel 111 to pass the gas products through a miniature catalytic converter 106 to convert C-containing and N-containing gas species to CO₂ and N₂, respectively. Spectral chemical sensors 107a-b detect a range of different nutrients such as N₂ and CO₂. The low-cost spectral sensor detects CO₂, different chemicals, or compounds in soil using electromagnetic spectrum analysis. This enables real-time analysis using a CO₂ sensor and a N₂ sensor, allowing direct determination of the C/N ratio based on measured CO₂ and N₂ concentration changes and corresponding flow rates. By combining soil density and the volume of the heating zone, SOC, and total soil N, are calculated based on the soil mass. Unlike other in-situ SOC sensing technologies, the provided embodiment does not require comparison with conventional lab methods or the need for a pre-calibrated prediction model, making it simpler and more accurate. The provided embodiment is portable, affordable (e.g., approximately $250 per sensing device), and fast (e.g., less than 10 minutes for one deep-soil test). With an integrated ultrasonic depth sensor and GPS sensor, the sensing device can profile SOC (and total N) and C/N ratio along the soil depth over a large area. Therefore, it can provide enriched data for decoding C/N dynamics, offering significant contributions to soil carbon and nitrogen measurements with high accuracy and spatiotemporal resolution. Several novel features across sensing technologies, soil science based on fundamental science, and engineering are configured to be seamlessly integrated into this novel sensing system. FIGS. 1A-1D illustrate schematic views of the proposed sensing system and its soil sampling method.

FIGS. 1B-1C illustrate the system 100 during different stages of insertion into soil 110. As illustrated in FIG. 1B, the system 100 can be pressed into the soil 110 using a hydraulic press 112. An exemplary implementation of this embodiment can be where a hand-held portable device, or a device mounted on an unmanned aerial vehicle (UAV), presses against an upper portion of the system 100 to lower the entire assembly to the required depth. The outer tube 108 has no soil in it at the insertion depth represented in FIG. 1B. FIG. 1C illustrates a second pressing of the tube outer 108 causing the outer tube 108 to go an additional distance, e.g., one or two inches, deeper into the soil while the inner tube 102 remains where it is. The soil then fills the gap between the inner tube and the outer tube. The volume and weight of the soil can be calculated from the diameter of the tube and the height of the gap between inner tube and outer tube.

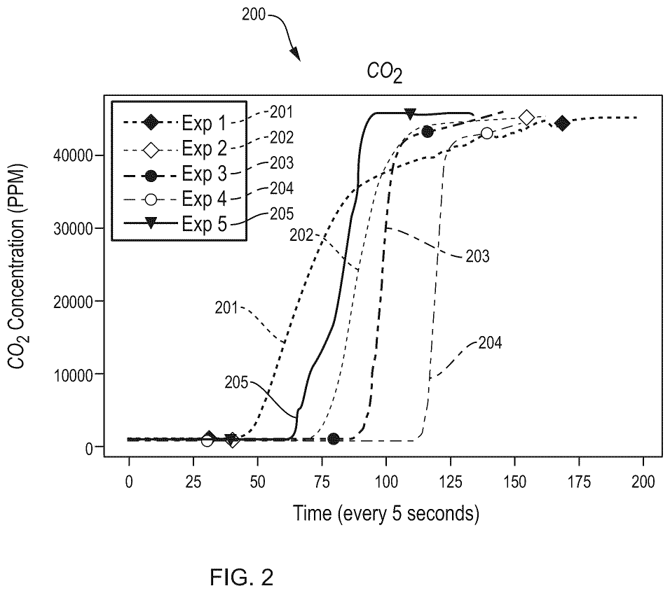

FIG. 1D illustrates the application of heat to the soil in the heating zone 103 via the heating rod 101. Applying heat to the soil causes the soil to gradually burn and generate organic matters 113, which are typically gaseous and which flow through the channel 111 and accumulate at or near the top of the outer tube 108. The optical based soil nutrient sensor 114 mounted on the top then measures the concentration. The sensor reading keep increasing as more and more organic matter accumulates from the soil. The sensor reading saturates when all the soil is completely burned. At this point, one can estimate the total organic matter from the initial and final concentrations reading. Example data showing estimated CO₂ concentrations as a function of time are shown in FIG. 2, as further described below.

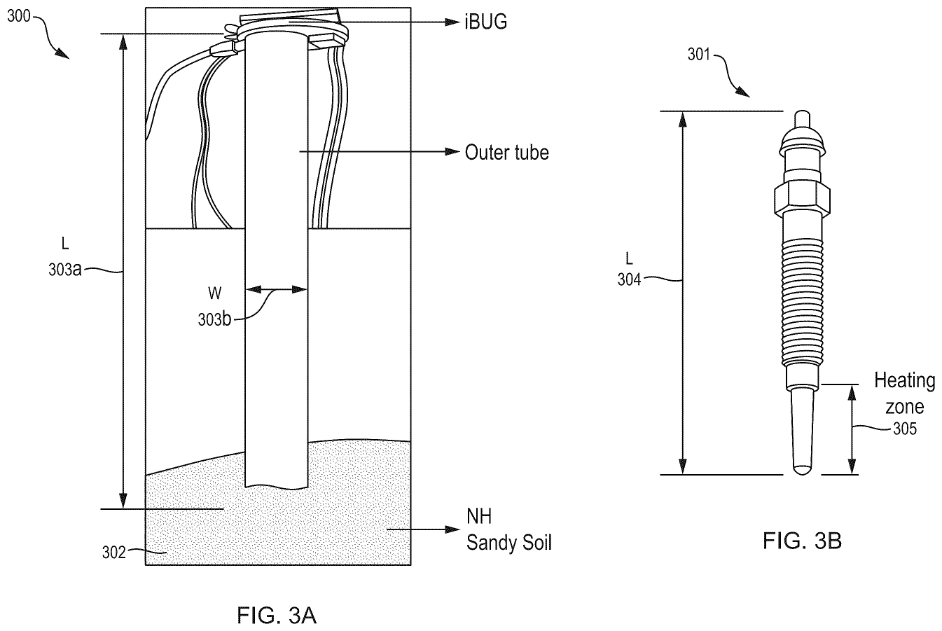

FIG. 3A shows a prototype version 300 of the exemplary embodiment of FIG. 1A, where the probe is inserted into the soil 302, as well as the dimensions 303a-b (length (e.g., 10 inches) and width (e.g., 1 inch) respectively) of the outer tube. FIG. 3B illustrates an example prototype heating element 301 for use with embodiments of the invention. FIG. 3B shows a dimension 304 for the length (e.g., 3 inches) of the heating element 301, and the heating zone location 305 of the heating element 301.

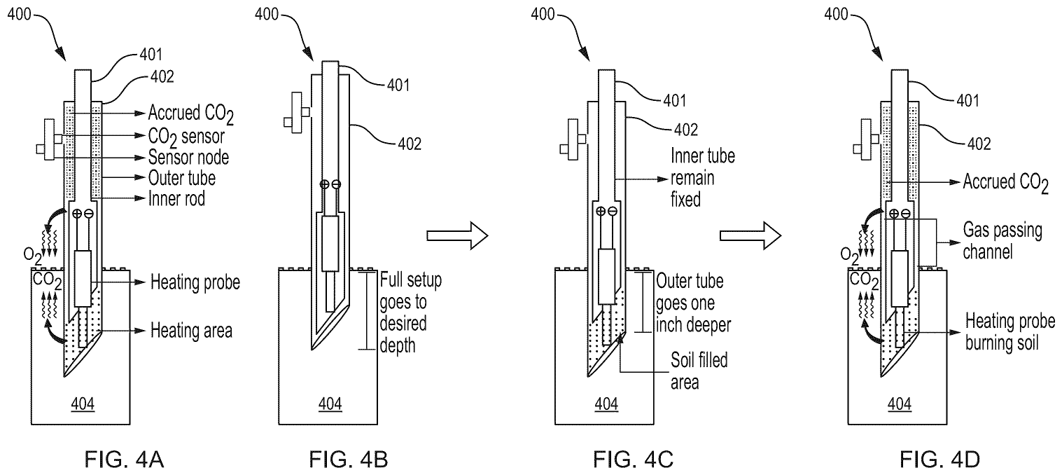

FIGS. 4A-4D illustrate an embodiment which is a cost-effective, high-precision sensing system 400 for detecting deep soil C/N with high spatiotemporal resolution. The system can employ a simple heating-based decomposition method combined with a catalytic converter and gas detectors in a double-walled tube platform. An example implementation of this embodiment can be where a hand-held portable device, or a device mounted on an unmanned aerial vehicle (UAV), presses against an upper portion of the system 400 to lower the entire assembly to the required depth. The outer tube 402 has no soil in it at the insertion depth represented in FIG. 4B. FIG. 4C illustrates a second pressing of the outer tube 402 causing it to go an additional distance, e.g., one or two inches, deeper into the soil while the inner tube 401 remains where it is. The soil then fills the gap between the inner tube 401 and the outer tube 402. The volume and weight of the soil 404 can be calculated from the diameter of the outer tube 402 and the height of the gap between inner tube 401 and outer tube 402.

FIG. 4D illustrates the application of heat to the soil in the heating zone via the heating rod. Applying heat to the soil 404 causes the soil 404 to gradually burn and generate organic matters, which are typically gaseous and which flow through the channel and accumulate at or near the top of the outer tube 402.

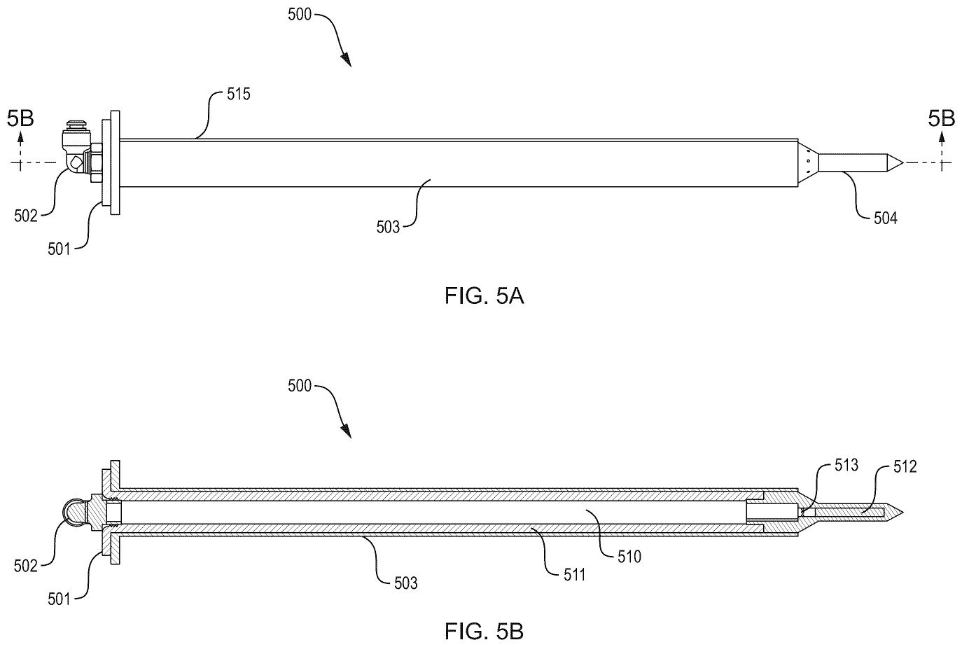

FIGS. 5A-5B illustrate a system for in-situ measurement of nutrients in soil according to an embodiment of the invention. System 500 includes an outer tube 503 (e.g., an outer pipe shell) and an inner tube 511 (e.g., an inner pipe shell). FIG. 5A shows the national pipe taper (NPT) fitting 502 atop the top portion 501 (e.g., flange) of the inner tube 511, as well as the outer tube 503 and the heating element contained within the tip 504. As illustrated, the outer tube 503 can include a longitudinal feature 515 (e.g., a guide track).

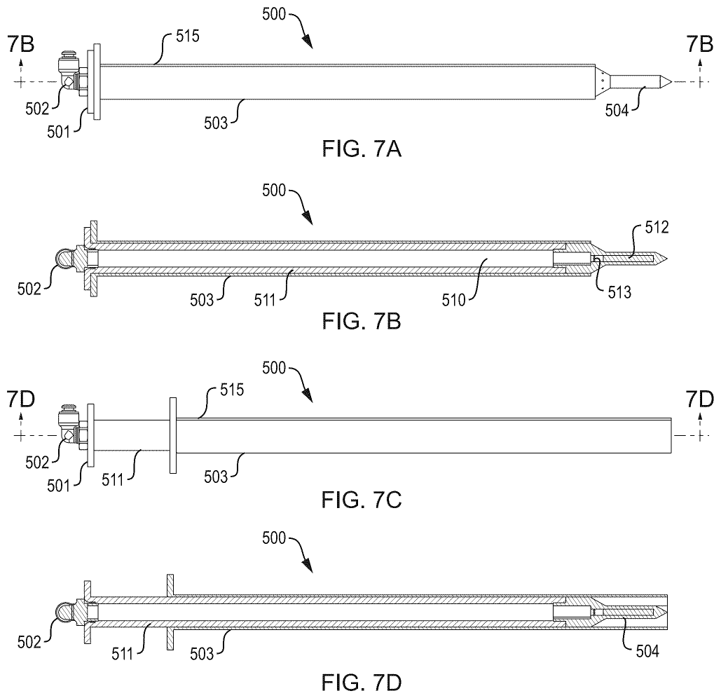

FIG. 5B illustrates a cross-sectional view of the system 500. The cross-sectional view in FIG. 5B shows the outer tube 503 as well as the inner tube 511. The inner tube 511 contains the carbon as it flows up through the center, as well as the wiring (not shown) for the heating device 512. The outer tube 503 is free fit around the inner tube 511 to allow the outer tube 503 to slide up and down on the inner tube 511 to enclose a volume of soil around the tip 504 containing the heating device 512. (See FIG. 7D) An NPT fitting 502 screws into the inner tube 511 to allow connection to a vacuum hose (not shown) to pull carbon from the soil and allow for routing of the heater wires (not shown). Holes 513 are drilled around the tip 504 to allow the carbon to flow into the probe and up through the center. The tip 504 screws into the inner tube 511 to allow for easy heater cartridge 512 access and cleaning. The heater cartridge 512 provides heating to the soil (not shown) to burn carbon out of the soil. In this embodiment, there is only a channel 510 for a return path for gaseous products, and there is no channel for the supply of air to the heating zone. There is oxygen present in the ground, particularly in the upper layers of soil. The hypothesis is that there is enough oxygen in the rooting zone of the soil, so that there is no need to supply additional oxygen to decompose the SOC or SON.

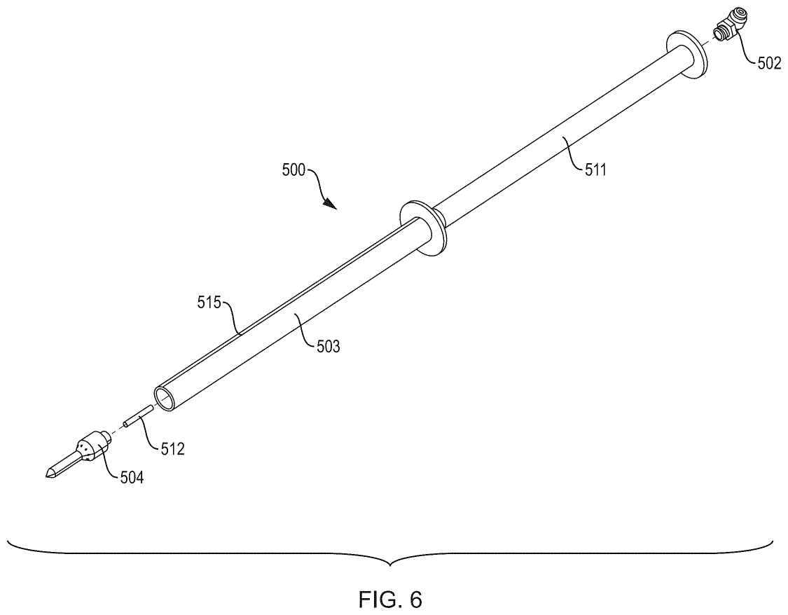

FIG. 6 shows an exploded perspective view of the system 500 of FIGS. 5A-5B illustrating how the various components of the system 500 may fit together. The system 600 as shown in FIG. 6 is comprised of the guide track 515, the tip 504, the heating cartridge 512, the outer tube 503, the inner tube 511, and the NPT fitting 502.

FIGS. 7A-7B show the system for in-situ measurement of nutrients in soil according to an embodiment of the invention in a standard configuration that includes the same components as shown in FIGS. 5A-5B in the same configuration. FIG. 7C-7D show the system 500 in a measuring configuration, where the outer tube 503 is moved downward along the inner tube 511 away from the NPT fitting 502 when the probe is at the desired measurement depth to enclose the volume of soil (not shown) around the heated tip 504 to capture carbon in the local area.

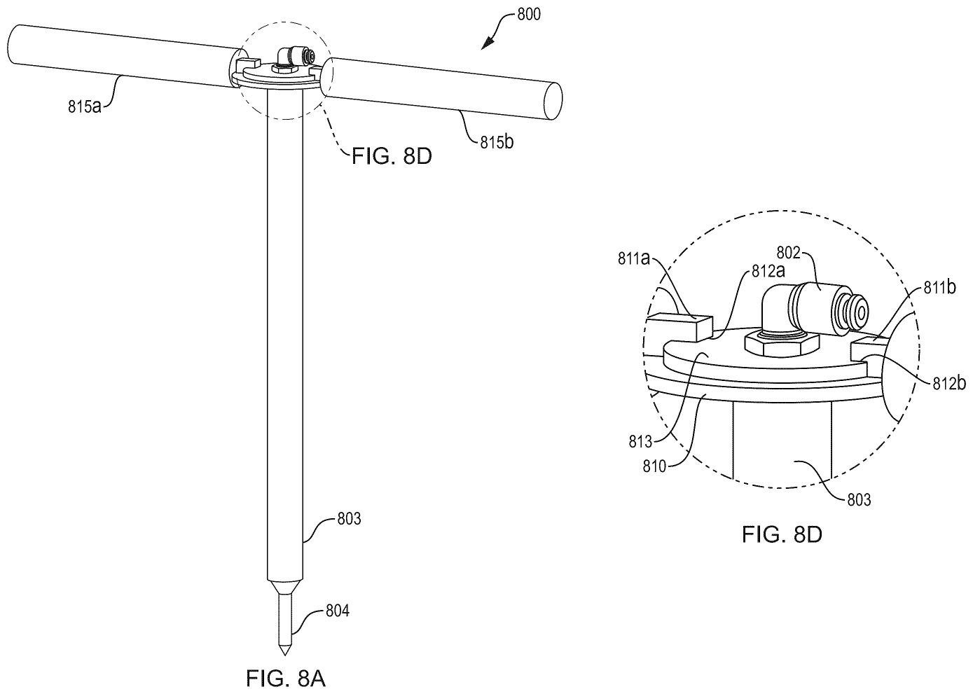

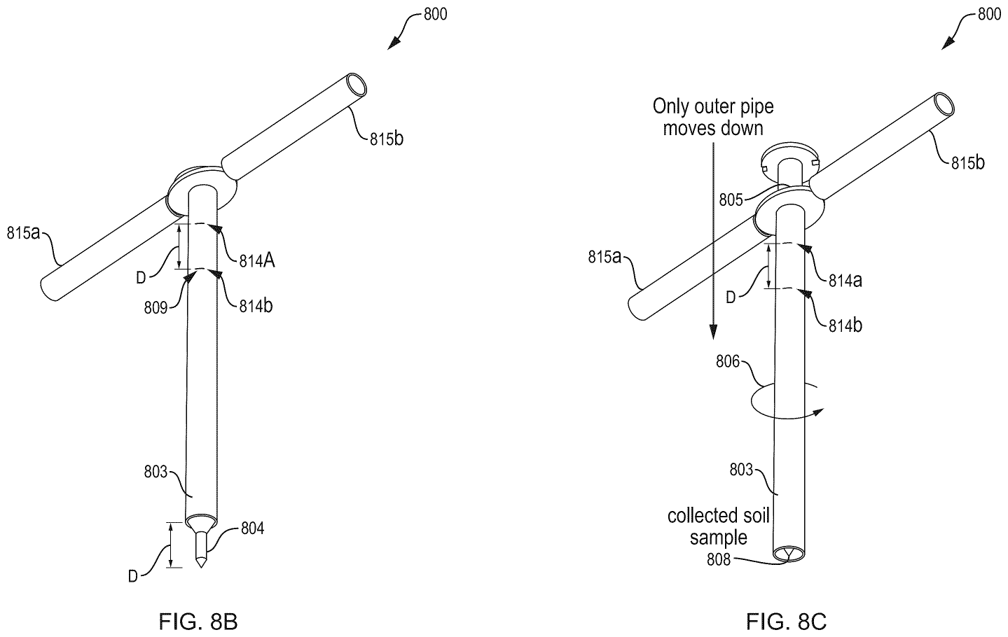

FIGS. 8A-8D illustrate an embodiment of the system 800 similar to that shown in FIGS. 5A-5B (i.e., NPT fitting 802, outer tube 803 and the tip 804) with the addition of two handles 815a, 815b and a locking mechanism 809 (FIG. 8B-8C), that are used to press the system 800 into the soil. FIG. 8D shows an enlarged portion of the top portion of the system. In this enlarged view, it is illustrated that the outer tube 803 has a flange 810 on its top portion. The flange 810 includes two tabs (protrusions) 811a-b. These tabs 811a and 811b fit into notches 812a and 812b, respectively, that are cut within a flange 813 of the inner tube 805 (FIG. 8C). This prevents the inner tube 805 from sliding through the outer tube 803, as well as preventing unwanted rotation between the inner tube and outer tube 803.

FIG. 8B shows an illustration of the system 800 in the first position as the system 800 is being pressed into the soil (not shown). In this position, the locking mechanism (including upper and lower slots 814a and 814b, respectively) is in its “first position” state. The upper and lower slots 814a and 814b are spaced apart by a distance “D” and the outer tube is moved by the same distance “D,” where the distance “D” is, for example, 2 inches. FIG. 8B illustrates that once the system 800 has been pressed into the appropriate soil depth (for example, 2 inches), either by the handles 815a, 815b or by another means, the outer tube 803 can be rotated relative to the inner tube 805 (shown by the arrow 806) counterclockwise along a channel (not shown) between the inner tube 805 and the outer tube 803. As the outer tube 803 is pushed down, the tabs 811a and 811b are released from the notches 812a and 812b, respectively, allowing the outer tube 803 to be rotated relative to the inner tube 805. This relative rotation 806 between the inner tube 805 and outer tube 803 helps to ensure that the soil sample is properly aligned with the tube's slot between the inner tube 805 and outer tube 803. This rotation 806 will continue until the outer tube 803 reaches a predefined final position (second position) defined by the upper slot 814a of the locking mechanism (for example, two inches) where the locking mechanism will lock the outer tube 803 into place. When the soil sample 808 is securely collected within the outer tube 803, a sensor (not shown) will initiate the heating process. As the heating process continues, the gases from the soil are drawn into the sensors detection system (see 107a, 107b and 114 of FIG. 1A) via a pump (not shown).

### Example: In-Situ Method for Measuring Soil Organic Carbon

For experimental testing of an embodiment a portable soil heating probe sensor, a steel tube of 10 inches in length and one inch in diameter was used. Four buckets of sandy soil samples from New Hampshire, US were collected. The tube fills up from the bottom when it goes into the soil. Consequently, the amount of soil is measured from the tube's depth. To burn the soil, a heating probe that could go up to 1000° C. was used. The heating probe was wired up to a programmable direct current (DC) power supply. The voltage was set to 9 volts, and the current was set to 5 amps. The probe's tip reached a temperature of approximately 400° C. at this setting, which was sufficient to decompose the OC in the soil.

For data collection, a sensor node named iBUG developed by the remote sensing lab of the University of New Hampshire was used. iBUG is a low-cost, TinyML and IoT-enabled sensing platform. It has a preinstalled SCD4x photoacoustic sensor that measures true CO₂. iBUG can process sensor data in real-time or transfer it to cloud using LoRa. The iBUG was placed at the top of the tube. It was then connected to a computer via a micro-USB cable to log the sensor readings. First, the sensor reading was taken at room temperature for a few minutes to measure the initial CO₂ Concentration. After some time, the heating probe was turned on, and the data collection process continued. The amount of soil that we considered for the experiment did not require more than five minutes to burn out. So, the heating probe was turned off after five minutes. Python was used to analyze the CO₂ concentration change trend once the data was collected. Some data points at the beginning and the end were discarded, as those were insignificant.

Concentrations of chemicals in soil are typically measured in units of the mass of chemical (grams(g), milligrams(mg), or micrograms(ug)) per mass of soil (kilogram, kg). Concentrations can be expressed as parts per million (ppm) using a conversion factor. Each chemical's conversion factor is based on its molecular weight. The CO₂ sensor used provides measurements in(ppm). It is converted to milligrams using the following equation C (mg/m\hat{ }3)=0.0409×C(ppm)×M Where, C is concentration in ppm; M is the molecular weight of CO₂ (44.01 g/mol).

The amount of SOC per unit area in a given depth of soil can be calculated using the following equation: SOC=% C×p×d. Where, SOC is soil organic carbon (g/m2); % C is carbon concentration; p is soil bulk density; d is depth of soil sample. Here, bulk density is the dry weight of a known amount of soil.

Five lab experiments were run on the same amount of soil (around 5 g) to verify for consistency. FIG. 2 shows plot 200 illustrating CO₂ concentration change over time as heat is applied in the five experiments 201-205. Before heating, the CO₂ concentration line is flat and indicate the normal CO₂ concentration. After the heating is turned on, the CO₂ concentration line starts to ascend rapidly, and this trend continued. After a certain amount of time has passed, the line begins to flatten once more. At this point, the concentration of CO₂ reaches its maximum level. When all CO₂ from soil sample comes out the sensor reading saturates. CO₂ concentration change is recorded from the final and initial saturation points converted to a common unit (g/Kg) to compare with soil weight loss shown in Table 1.

TABLE 1

| | Soil | Soil | Accrued | SOC from | SOC from |
|---|---|---|---|---|---|
| | Weight | Weight | CO₂ | LOI | Sensor |
| | Loss (g) | Loss (g/kg) | (g/kg) | (g/m²) | (g/m²) |
| EXP 1 | 0.31 | 54.87 | 43.06 | 49.38 | 48.75 |
| EXP 2 | 0.25 | 55.31 | 44.15 | 49.78 | 49.74 |
| EXP 3 | 0.27 | 53.78 | 41.93 | 48.41 | 47.74 |
| EXP 4 | 0.34 | 58.72 | 44.84 | 52.85 | 50.36 |
| EXP 5 | 0.32 | 59.93 | 44.5 | 53.93 | 50.05 |

The fourth column in Table 1 shows the accrued CO₂ concentration converted using the equation C (mg/m\hat{ }3)=0.0409×C(ppm)×M. Then, the OC percentage was calculated from the fourth column to calculate the SOC using the equation SOC=% C×p×d. The soil weight loss was calculated by weighing the soil before and after heating. The weight loss occurred due to the evaporation of the OC from the soil. OC percentage was calculated from this weight loss, and then the equation C (mg/m\hat{ }3)=0.0409×C(ppm)×M was used to estimate SOC from LOI. SOC estimates from weight loss corresponds to the results of heating-probe sensor. In addition to SOC, water particles and some other soil constituents come through soil combustion. These components also contribute to soil weight loss. As a result, there is a corresponding difference between LOI and CO₂ sensor estimates. However, this is offset by adding a value of 10 in the final result.

Lab experiments show promising results, such as those presented in FIG. 2, that can be applied in the field. Comparing field results to results obtained with existing methodology is useful to validate the new approach. Additionally, it is expected that iBUG's machine learning capacity can be used for real-time SOC prediction. During data collection, temperature, humidity, and gas resistance can be recorded. Such data is expected to enable development of a machine learning model to predict CO₂ concentration.

### REFERENCES

[1] J. H. Sato, C. C. de Figueiredo, R. L. Marchão, B. E. Madari, L. E. C. Benedito, J. G. Busato, and D. M. de Souza, “Methods of soil organic carbon determination in Brazilian savannah soils,” Scientia Agricola, vol. 71, no. 4, pp. 302-308, August 2014.
[2] X. Yu, W. Zhou, Y. Wang, P. Cheng, Y. Hou, H. Du, X. Xiong, L. Yang, Y. Wang, and Y. Fu, “Sequential combustion separation of soil organic carbon fractions for AMS measurement of 14C and their application in fixation of carbon,” Journal of Radioanalytical and Nuclear Chemistry, vol. 323, no. 1, pp. 169-177, January 2020.
[3] M. Hoogsteen, E. Lantinga, E. Bakker, and P. Tittonell, “An Evaluation of the Loss-on-Ignition Method for Determining the Soil Organic Matter Content of Calcareous Soils,” Communications in Soil Science and Plant Analysis, vol. 49, no. 13, pp. 1541-1552 July 2018.
[4] T. K. Boguski, “Understanding Units of Measurement,” Environmental Science and Technology Briefs for Citizens, p. 2, 2006.
[5] S. R. Abella and B. W. Zimmer, “Estimating Organic Carbon from Loss-On-Ignition in Northern Arizona Forest Soils,” Soil Science Society of America Journal, vol. 71, no. 2, pp. 545-550, March 2007.
[6] W. R. Roper, W. P. Robarge, D. L. Osmond, and J. L. Heitman, “Comparing Four Methods of Measuring Soil Organic Matter in North Carolina Soils,” Soil Science Society of America Journal, vol. 83, no. 2, pp. 466-474, 2019.
[7] T. Angelopoulou, A. Balafoutis, G. Zalidis, and D. Bochtis, “From Laboratory to Proximal Sensing Spectroscopy for Soil Organic Carbon Estimation-A Review,” Sustainability, vol. 12, no. 2, p. 443, January 2020.
[8] M. Ladoni, H. A. Bahrami, S. K. Alavipanah, and A. A. Norouzi, “Estimating soil organic carbon from soil reflectance: A review,” Precision Agriculture, vol. 11, no. 1, pp. 82-99, February 2010.
[9] X. Peng, T. Shi, A. Song, Y. Chen, and W. Gao, “Estimating Soil Organic Carbon Using VIS/NIR Spectroscopy with SVMR and SPA Methods,” Remote Sensing, vol. 6, no. 4, pp. 2699-2717 April 2014.
[10] N. K. Wijewardane, S. Hetrick, J. Ackerson, C. L. Morgan, and Y. Ge, “Visnir integrated multi-sensing penetrometer for in situ high-resolution vertical soil sensing,” Soil and Tillage Research, vol. 199, p. 104604, 2020. [Online]. Available: https://www.sciencedirect.com/science/article/pii/S0167198719305136
[11] M. F. Yousuf, T. Siddique, and M. S. Mahmud, “iBUG: AI Enabled IoT Sensing Platform for Real-time Environmental Monitoring,” in 2022 IEEE 31st Microelectronics Design & Test Symposium (MDTS), May 2022, pp. 1-7.
[12] (2021) Sensirion co2 sensors scd4x datasheet. [Online]. Available: https://www.mouser.com/datasheet/2/682/
[13] R. Zeng, Y. Wei, J. Huang, X. Chen, and C. Cai, “Soil organic carbon stock and fractional distribution across central-south China,” International Soil and Water Conservation Research, vol. 9, no. 4, pp. 620-630, December 2021.
[14] M. F. Yousuf, M. S. Mahmud, B. Li, Y. Lei and H. Tao, “A Novel In-situ Method for Measuring Soil Organic Carbon Using Photoacoustic Sensor,” 2022 IEEE Sensors, Dallas, TX, USA, 2022, pp. 1-4, doi: 10.1109/SENSORS52175.2022.9967116.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A system for in-situ measurement of substances in soil, the system comprising:
a probe comprising an outer portion and an inner portion;
the inner portion movably coupled to the outer portion and positioned inside the outer portion;
the outer portion comprising:
a lower end adapted for insertion into soil, and for isolating a soil sample in a heating zone;
one or more gaseous fittings arranged to provide gas to the heating zone, or to remove gas from the heating zone, or both;
the inner portion being of a smaller dimension in width than the outer portion, and the inner portion and the outer portion arranged to provide one or more channels for the passage of air, gaseous products, or both;
a heating device configured to apply heat to the soil sample in the heating zone to release gaseous products; and
a sensor arranged to detect substances in the gaseous products released, the substances being indicative of nutrients in the soil.

2. The system of claim 1, wherein the outer portion is a cylindrical tube, and the inner portion is a cylindrical rod.

3. The system of claim 1, wherein both the outer portion and the inner portion are cylindrical tubes.

4. The system of claim 1, further comprising a portable power supply configured to supply power the heating device.

5. The system of claim 1, wherein the system is integrated in a portable hand-held device.

6. The system of claim 1, wherein the system is mounted on an unmanned aerial vehicle, and further including a hydraulic press to insert the system into the soil.

7. The system of claim 1, wherein the inner portion is of a smaller dimension than the outer portion, such that a channel is created between the inner portion and the outer portion for the passage of air and gaseous products.

8. The system of claim 1, wherein a gaseous inlet and a gaseous outlet are positioned such that a channel between the gaseous inlet and the heating zone is created, and a separate channel between the heating zone and the gaseous outlet is created.

9. The system of claim 8, further comprising a mesh screen attached radially around the inner portion above the heating device such that the mesh screen creates a barrier preventing the soil from entering the channels between the inlet and the heating zone, and the heating zone and the outlet.

10. The system of claim 8, further comprising a catalyst converter operably coupled to the gaseous outlet.

11. The system of claim 10, wherein the catalyst converter converts Carbon-containing gas species and Nitrogen-containing gas species in the gas products into CO₂ and N₂, respectively.

12. The system of claim 11, further comprising a sensor node operably coupled to the catalyst converter, the sensor node including the sensor arranged to detect the substances in the gas products and an environmental sensor arranged to detect CO₂ content in air.

13. The system of claim 12, wherein the sensor node is configured to detect an amount of CO₂ and an amount of N₂.

14. The system of claim 1, wherein the inner and outer portions are movable between a first position and a second position, wherein:
in the first position, the system is insertable into the soil utilizing the lower end of the outer portion such that the outer portion and the inner portion are both at the same depth in the soil;
in the second position, the outer portion is extendable deeper into the soil, leaving the inner portion at a shallower depth in the soil and capturing the soil sample around the heating device; and
an upper end of the inner portion extending through an opening at a top end of the outer portion as the outer portion extends deeper into the soil.

15. The system of claim 14, wherein the inner portion extending through the opening at the top surface of the outer portion has markings on it which indicate a difference in depth between the outer portion and inner portion, such that a volume of the soil filled area can be calculated.

16. The system of claim 14, further comprising a locking mechanism coupled to the inner portion and the outer portion and configured to lock the system in the first position or the second position.

17. A method for in-situ measurement of substances in soil, the method comprising:
inserting a probe into soil;
isolating a soil sample in a heating zone;
applying heat to the soil sample in the heating zone to release gas products; and
detecting substances in the gas products released, the substances being indicative of nutrients in the soil.

18. The method of claim 17, wherein the probe comprises an inner portion movably coupled to any position inside an outer portion,
the inner portion movably coupled to the outer portion and positioned inside the outer portion;
the outer portion comprising:
a lower end adapted for insertion into the soil, and for capturing the soil sample in the heating zone; and
one or more gaseous fittings arranged to provide gas to the heating zone, or to remove gas from the heating zone, or both.

19. The method of claim 18, wherein the inner portion is of a smaller dimension than the outer portion, such that a channel is created between the inner portion and the outer portion for the passage of air and gaseous products.

20. The method of claim 18, wherein inserting the probe into the soil includes inserting the inner and outer portions of the probe into the soil to a first depth, and further inserting the outer portion into the soil to a second depth.

21. The method of claim 20, wherein at least one of the inner portion and the outer portion is inserted into the soil utilizing a hydraulic press.

22. The method of claim 20, wherein detecting the substances in the gas products includes catalytically converting Carbon-containing gas species and Nitrogen-containing gas species in the gas products into CO₂ and N₂, respectively, and detecting the CO₂ and N₂.

23. The method of claim 18, wherein the inner and outer portions are movable between a first position and a second position, wherein:
in the first position, inserting the system into the soil utilizing the lower end of the outer portion such that the outer portion and the inner portion are both at the same depth in the soil;
in the second position, extending the outer portion deeper into the soil, leaving the inner portion at a shallower depth in the soil and capturing the soil sample around the heating device; and
extending an upper end of the inner portion through an opening at a top end of the outer portion as the outer portion extends deeper into the soil.

24. The method of claim 23, further comprising locking the system in the first position or the second position using a locking mechanism coupled to the inner portion and the outer portion.
